(19) United States
(12) Patent Application Publication
(10) Pub. No.: US 2025/0235289 A1
(43) Pub. Date: Jul. 24, 2025

(54) MARKER REFLECTOR SYSTEMS WITH LIGHT EMITTERS

(71) Applicant: Cianna Medical, Inc., Aliso Viejo, CA (US)

(72) Inventors: Eduardo Chi Sing, Dana Point, CA (US); John E. Greene, Valley Center, CA (US); MIke Lopez, Aliso Viejo, CA (US); Nikolai Rulkov, San Diego, CA (US)

(21) Appl. No.: 19/033,821

(22) Filed: Jan. 22, 2025

Related U.S. Application Data
(60) Provisional application No. 63/624,198, filed on Jan. 23, 2024.

(51) Int. Cl. A61B 90/00

(52) U.S. Cl. CPC A61B 90/39; A61B 2090/3908; A61B 2090/3945; A61B 2090/3979

(57) ABSTRACT

Apparatus, systems, and methods are provided for localizing lesions within a patient's body, e.g., within a breast. The system may include one or more markers implantable within or around the target tissue region, and a probe for transmitting and receiving electromagnetic signals to detect the one or more markers. The marker(s) may be placed within a target tissue region, and the probe may be placed against the patient's skin to detect and localize the marker(s). The marker(s) may include light emitting diodes to provide a visual indication.

### RELATED APPLICATIONS

This application claims priority to U.S. Provisional Application No. 63/624,198, filed on Jan. 23, 2024 and titled, “Marker Reflector Systems with Light Emitters,” which is hereby incorporated by reference in its entirety.

### TECHNICAL FIELD

The present disclosure relates to implantable tags or markers and to systems and methods for identifying and/or locating multiple markers within a patient's body, e.g., during surgical procedures or other procedures, such as during lumpectomy procedures.

### BRIEF DESCRIPTION OF THE SEVERAL VIEWS OF THE DRAWINGS

The written disclosure herein describes illustrative embodiments that are non-limiting and non-exhaustive. Reference is made to certain of such illustrative embodiments that are depicted in the figures, in which:

FIG. 1 shows an exemplary embodiment of a system for localization of a target tissue region within a patient's body in accordance with some embodiments.

FIG. 2 shows an exemplary embodiment of the system for localization of a target tissue region within a patient's body that includes a probe and a plurality of reflectors, tags, or markers in accordance with some embodiments.

FIG. 3 is a block diagram showing exemplary components of a controller of the probe in accordance with some embodiments.

FIG. 4 illustrates a block diagram of a marker in accordance with some embodiments.

FIG. 5A illustrates a circuit diagram of one possible embodiment of the marker wherein a relaxation oscillator may be utilized to pulse a Light Emitting Diode (LED).

FIG. 5B illustrates a response of a switch of the circuit diagram of the marker with a relaxation oscillator in accordance with some embodiments.

FIG. 6 illustrates a perspective view of an up conversion device in accordance with some embodiments.

FIG. 7 is a side view of a probe localizing a plurality of markers implanted within a breast in accordance with some embodiments.

FIG. 8 illustrates a circuit diagram of one possible embodiment of a marker where an LED is connected to a power harvesting module of an Application Specific Integrated Circuit (ASIC) reflector.

FIG. 9 illustrates a block diagram of one possible embodiment of a marker where a LED is connected to a Power Harvesting module and may produce visible light as an indication to the user.

FIG. 10 illustrates a circuit diagram of one possible embodiment of a marker where orthogonal codes may be used for LED modulation and localization of the LED of the marker.

### DETAILED DESCRIPTION

Before a biopsy or surgical procedure to remove a lesion within a breast, e.g., during a lumpectomy procedure, the location of the lesion must be identified. For example, mammography or ultrasound imaging may be used to identify and/or confirm the location of the lesion before the procedure. The resulting images may be used by a surgeon during the procedure to identify the location of the lesion and guide the surgeon, e.g., during dissection to access and/or remove the lesion. However, such images are generally two dimensional and therefore provide only limited guidance for localization of the lesion since the breast and any lesion to be removed are three-dimensional structures. Further, such images may provide only limited guidance in determining a proper margin around the lesion, i.e., defining a desired specimen volume to be removed.

To facilitate localization, immediately before a procedure, a wire may be inserted into the breast, e.g., via a needle, such that a tip of the wire is positioned at the location of the lesion. Once the wire is positioned, it may be secured in place, e.g., using a bandage or tape applied to the patient's skin where the wire emerges from the breast. With the wire placed and secured in position, the patient may proceed to surgery, e.g., to have a biopsy or lumpectomy performed.

One problem with using a wire for localization is that the wire may move between the time of placement and the surgical procedure. For example, if the wire is not secured sufficiently, the wire may move relative to the tract used to access the lesion and consequently the tip may misrepresent the location of the lesion. If this occurs, when the location is accessed and tissue removed, the lesion may not be fully removed and/or healthy tissue may be unnecessarily removed. In addition, during the procedure, the surgeon may merely estimate the location of the wire tip and lesion, e.g., based on mammograms or other images obtained during wire placement, and may proceed with dissection without any further guidance. Again, since such images are two dimensional, they may provide limited guidance to localize the lesion being treated or removed.

Alternatively, it has been suggested to place a radioactive seed to provide localization during a procedure. For example, a needle may be introduced through a breast into a lesion, and then a seed may be deployed from the needle. The needle may be withdrawn, and the position of the seed may be confirmed using mammography. During a subsequent surgical procedure, a hand-held gamma probe may be placed over the breast to identify a location overlying the seed. An incision may be made and the probe may be used to guide excision of the seed and lesion.

Because the seed is delivered through a needle that is immediately removed, there is risk that the seed may migrate within the patient's body between the time of placement and the surgical procedure. Thus, similar to using a localization wire, the seed may not accurately identify the location of the lesion, particularly, since there is no external way to stabilize the seed once placed. Further, such gamma probes may not provide desired precision in identifying the location of the seed, e.g., in three dimensions, and therefore may only provide limited guidance in localizing a lesion.

Accordingly, apparatus and methods for localization of lesions or other tissue structures in advance of and/or during surgical, diagnostic, or other medical procedures would be useful.

Embodiments herein are directed to implantable tags or markers, and to systems and methods for identifying and/or locating multiple markers within a patient's body, e.g., during surgical procedures or other procedures, such as during lumpectomy procedures.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

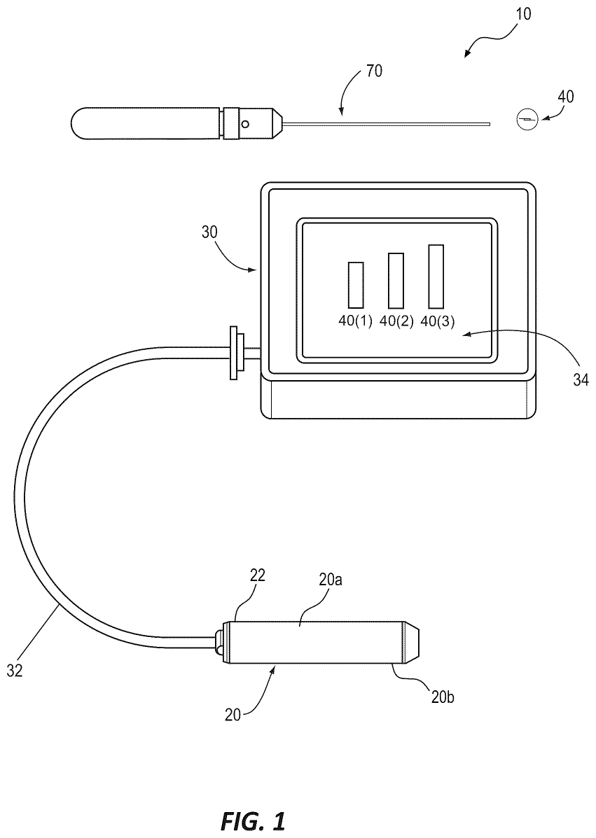

Turning to the drawings, FIG. 1 shows an exemplary embodiment of a system 10 for localization of a target tissue region within a patient's body that includes a probe 20 and a plurality of tags or markers 40 that may be implanted within a patient's body, e.g., within a target tissue region, such as within a breast.

In some embodiments, the markers 40 may comprise passive Radio-frequency identification (RFID) tags or active RFID tags. In some embodiments, the markers 40 may be configured to reflect and modulate a signal transmitted by the probe 20. In some embodiments, the probe 20 may transmit an RFID signal to identify the markers 40.

In other embodiments, the probe may transmit signals using ultra-wideband (UWB) radar. The markers 40 may reflect and modulate the UWB radar. The probe 20 may use reflected signals to identify the marker 40.

In some embodiments, the system 10 may include one or more additional components, e.g., one or more delivery devices 70, each carrying one or more reflectors, tags, or markers 40 (one shown) for introduction/implantation in a patient's body, and a controller and/or display unit 30 coupled to the probe 20, e.g., using one or more cables 32, similar to embodiments described in the applications incorporated by reference herein.

The probe 20 may include one or more antennas for transmitting signals using electromagnetic pulses, waves, or other signals, such as radar into a patient's body and receiving reflected signals from the patient's body. In addition, the probe 20 may include a light transmitter (e.g., a plurality of light fibers), configured to transmit light into tissue contacted by the distal end of the probe 20.

In some embodiments, the light source is an infrared light source, e.g., capable of delivering near infrared light between, for example, eight hundred and nine hundred fifty nanometers (800-950 nm) wavelength. The light source may include one or more lenses, and/or one or more filters (not shown). The lenses or filters may be used to focus the light transmitted by the probe 20 in a desired manner (e.g., in a relatively narrow beam or in a wider angle beam), and/or cause the light source to emit a specific band of light. In some embodiments, multiple light sources and/or filters may be provided to allow the probe 20 to deliver light pulses in different narrow bands.

The probe 20 may be a portable device having electromagnetic signal emitting and receiving capabilities (e.g., a RFID probe). The probe 20 may be a handheld device including a first or proximal end 20a configured to be held by a user, and a second or distal end 20b configured to be placed against or adjacent tissue, e.g., a patient's skin or underlying tissue.

The probe 20 may include one or more processors within its housing or within the display unit 30 including one or more controllers, circuits, signal generators, gates, and the like (not shown) needed to generate signals for transmission by transmit antennas and/or to process signals received from receive antennas. The components of the processor(s) may include discrete components, solid state devices, programmable devices, software components, and the like, as desired. Optionally, the probe 20 and/or display unit 30 may include other features or components, such as one or more user interfaces, memory, transmitters, receivers, connectors, cables, power sources, and the like. In addition, the processor(s) may be coupled to a display 34 of the display unit 30 for displaying information to a user of the probe 20, e.g., spatial or image data obtained using the probe 20.

The system 10 may be used during a medical procedure, to identify and locate a plurality of tags or markers 40 implanted within a patient's body. For example, in a breast biopsy or lumpectomy procedure, the markers 40 may be used to facilitate localization of a lesion or other target tissue region and/or to facilitate dissection and/or removal of a specimen from a breast. It should be noted that, although the system may also be used in localization of other objects in other areas of the body.

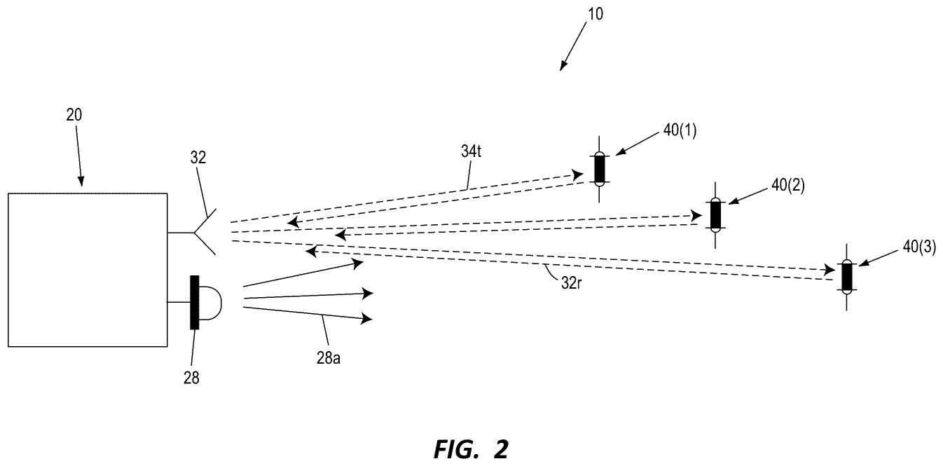

FIG. 2 shows an exemplary embodiment of the system 10 for localization of a target tissue region within a patient's body that includes a probe 20 and a plurality of reflectors, tags, or markers 40 (three shown merely for illustration) that may be implanted within a patient's body.

As shown, the probe 20 generally includes one or more antennas 22 for transmitting electromagnetic signals 34T into a patient's body and receiving reflected signals 34R from the patient's body, and a light source 28 for delivering light 28a into a patient's body synchronized with the electromagnetic signals. In an exemplary embodiment, the antenna(s) 22 transmit ultrawide band (UWB) radar pulses that are used for simultaneous detection of the markers 40 based on modulated reflective properties, e.g., using a switch inside each marker 40, which is controlled by light pulses from the light source 28. In other embodiments, the antenna(s) 22 may transmit an RFID signal and receive backscatter to detect and identify the markers.

In some embodiments, the light 28a from the light source 28 may be harvested, converted into electrical energy, and used by the markers 40 to power various components of the markers. For example, as previously described, the electrical energy may be used to alter a state of a switch inside each marker to alter the reflective properties of the marker. In some embodiments, the electrical energy from the light 28a of the light source 28 may power an electromagnetic transmission from the markers.

Additionally, in some embodiments, the markers may include a light emitting diode (LED) that may illuminate to provide a surgeon a visual indicator of the position and/or status of the marker. The LED may be powered by the electrical energy from the light 28a of the light source 28.

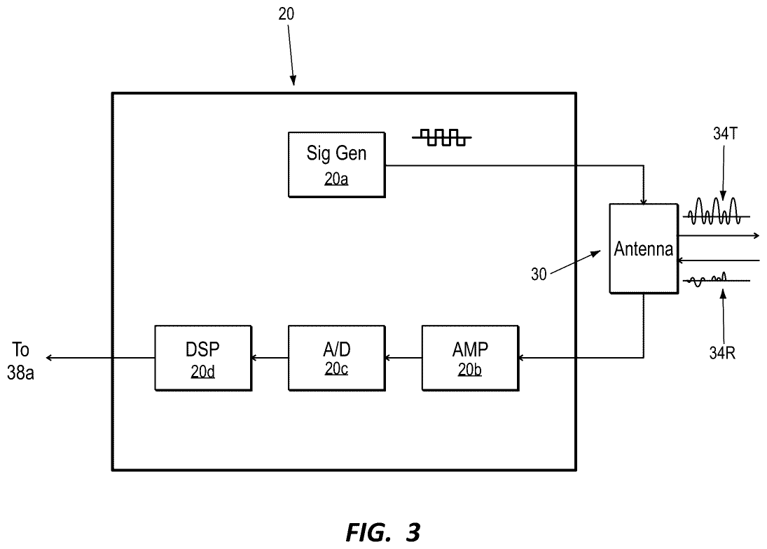

FIG. 3 is a block diagram showing exemplary components of a controller of the probe 20 (although, alternatively, some of the components may be located within the controller/display unit 30 of FIG. 1) in accordance with some embodiments. In the example shown, the probe 20 may include a signal generator 20a, an amplifier 20b, an analog-to-digital (A/D) converter 20c, and a digital signal processor (DSP) 20d. The signal generator 20a, e.g., a reference oscillator, produces an oscillating signal, such as a square wave signal, a triangular wave signal, or a sinusoidal signal.

For example, the probe 20 may include an impulse generator, e.g., a pulse generator and/or pseudo noise generator (not shown), coupled to the transmit antenna to generate transmit signals, and an impulse receiver for receiving signals detected by the receive antenna. The probe 20 may include a micro-controller and a range gate control that alternately activate the impulse generator and impulse receiver to transmit electromagnetic pulses, waves, or other signals via the transmit antenna, and then receive any reflected electromagnetic signals via the receive antenna, e.g., similar to other embodiments herein. Exemplary signals that may be used include microwave, radio waves, such as micro-impulse radar signals, e.g., in the ultrawide bandwidth region.

In the example shown in FIG. 3, a square wave signal may be sent from the signal generator 20a to the transmit antenna(s) 32T of the antenna assembly 30 of the probe 20. The antenna assembly may include a transmit antenna and a receive antenna. In some embodiments, the antenna elements may include a bowtie transmit antenna and a bowtie receive antenna with the transmit antenna offset ninety degrees (90°) from the receive antenna to define a Maltese cross antenna.

When the square wave signal passes through the transmit antenna(s) 32T, the transmit antenna(s) 32T may act as a band pass filter (“BPF”) and convert the square wave signal to a series of pulses or other transmit signals 34T. As such, the transmit signals 34T (shown in FIG. 3) transmitted by the probe 20 may include a series of pulses. Alternatively, the probe 20 may be configured to transmit continuous wave signals, e.g., similar to embodiments described in the references incorporated by reference herein.

The transmit signals 34T may be transmitted into the tissue and reflected from the implanted marker(s) 40, as represented by the receive signals 34R. Once the transmit signals 34T are reflected from the marker(s) 40, the reflected signals (i.e., the receive signals 34R) include a series of attenuated pulses (shown in FIG. 2).

As shown in FIGS. 2 and 3, the receive antenna(s) 32R of the antenna assembly 30 of the probe 20 may receive the receive signals 34R, which may be inputted into amplifier 20b in order to amplify the gain of the pulses. The output of the amplifier 20b may be inputted into an A/D converter 20c in order to convert the amplified analog signal into a digital signal. The digital signals output from the A/D converter 20c may be inputted into a DSP 20d for further processing. The DSP 20d may perform a number of processing functions including, but not limited to, calculating a difference in time from the time the transmit signals 34T were sent to the time the receive signals 34R were received (propagation time delay), determining the distance from the distal end 24 of the probe 20 to the marker 40, determining the location of the marker 40 relative to the distal end 24 of the probe 20, measuring the amplitude of the receive signals 34R, and/or determining the direction the marker 40 relative to the distal end 24 of the probe 20. For example, the probe 20 may determine distance from the distal end 24 of the probe 20 to the marker based on propagation time delay. The probe 20 may determine the location of the marker 40 relative to the distal end 24 of the probe by determining the distance at multiple locations, and using the distance measurements and the coordinates of the multiple locations to calculate a location of the marker 40.

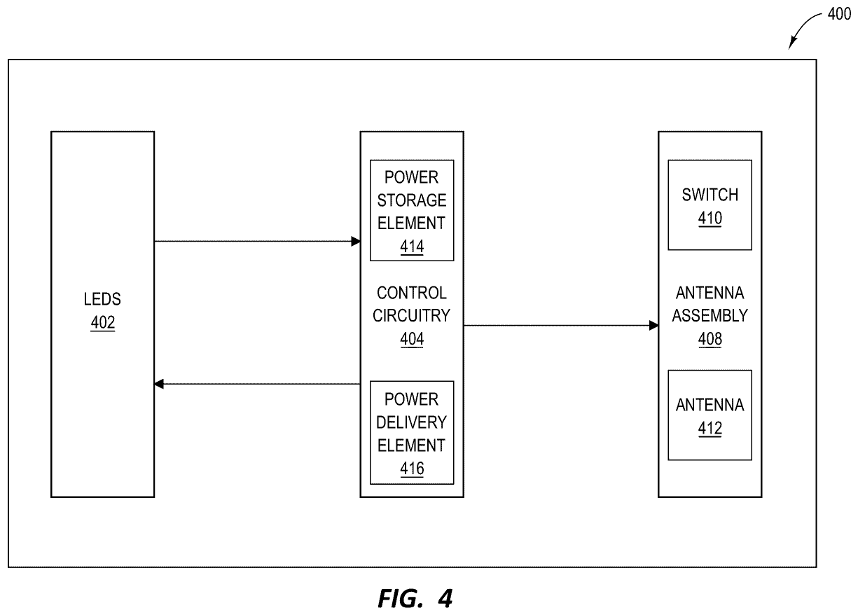

FIG. 4 illustrates a block diagram of a possible marker 400 embodiment comprising of a set of photosensitive diodes 401, one or more LEDs 402, a control circuitry 404, a 406, an antenna assembly 408, a switch 410, an antenna 412, a power storage element 414, and a power delivery element 416. The set of photosensitive diodes 401 may be configured to convert light received from a light source outside a patient's body into electrical energy which may be stored within the power storage element 414. In some embodiments, the power storage element 414 may be a capacitor.

In some embodiments, the control circuitry 404 may be configured to selectively apply electrical energy through the power delivery element 416 to one or more elements of the marker 400. For instance, the control circuitry 404 may selectively send electrical energy to the LEDs 402 to cause the one or more LEDs 402 to emit light. In some embodiments, the light may be used as a visual indicator to indicate that the marker 400 is operating correctly. The light may be used as visual indication for a surgeon as to the location of the markers that have been placed into the patient's body. In some embodiments, the light may be used to identify different markers. For instance the LEDs 402 for each marker may use different wavelengths, colors, flashing pattern, etc.

The control circuitry 404 may also selectively apply the electrical energy through the power delivery element 416 to the switch 410 to cause the switch 410 to open and close. In some embodiments, the control circuitry 404 may also send electrical energy to the antenna 412 to cause the antenna to emit a signal.

In some embodiments, at least some of the photosensitive diodes 401 used to convert light energy received from a light source to electrical energy may be LEDs. In some embodiments, the LEDs used for the photosensitive diodes 401 may be different than the one or more LEDs 402 used to emit the light. In the illustrated embodiment, the photosensitive diodes 401 that convert the external light to energy are separate from the LEDs 402 that emit the light.

However, in other embodiments, the one or more LEDs 402 that are used to emit the light may be a subset of the photosensitive diodes 401. For instance, in embodiments where at least a portion of the photosensitive diodes 401 are LEDs, the LEDs may supply the power storage element 414 with energy converted from the external light source when in a reverse biased configuration. The control circuitry 404 may selectively apply the electrical energy stored in the power storage element 414 across a subset of the LEDs (e.g., one of the LEDs) in a forward biased configuration when the capacitor reaches a threshold to cause the subset of LEDs to emit a light.

In certain embodiments, the control circuitry 404 comprises a relaxation oscillator within the control circuitry 404, coupled to the LEDs 402. The relaxation oscillator may provide a repetitive and periodic power output. In certain embodiments, the control circuitry 404 is configured such that the light pulses from the light source cause the switch 410 to open and close to modulate electromagnetic signals from a probe reflected by the marker. The switch may be open or closed for a certain duration depending on but is not limited to the duration of the light pulses, the duration between consecutive light pulses etc.

In certain embodiments, the control circuitry 404 may comprise a storage circuit including a power storage element 414 coupled to the photosensitive diodes 401. The power storage element 414 may store electrical energy generated by the photosensitive diodes 401 until a predetermined threshold is achieved. The storage circuit may be coupled to the switch 410 and provide power for closing the switch when the predetermined threshold is achieved to deliver electrical energy from the power delivery element 416 to elements of the marker such as the antenna 412, the switch 410, and the LEDs 402. In some embodiments, the power delivery element delivers electrical energy to the antenna, whereupon the antenna transmits a radio frequency (RF) signal. In certain embodiments, the control circuitry 404 comprises a sequence generator. The sequence generator may be coupled to the switch 410 to open and close the switch 410 to modulate backscatter signals based on a code sequence.

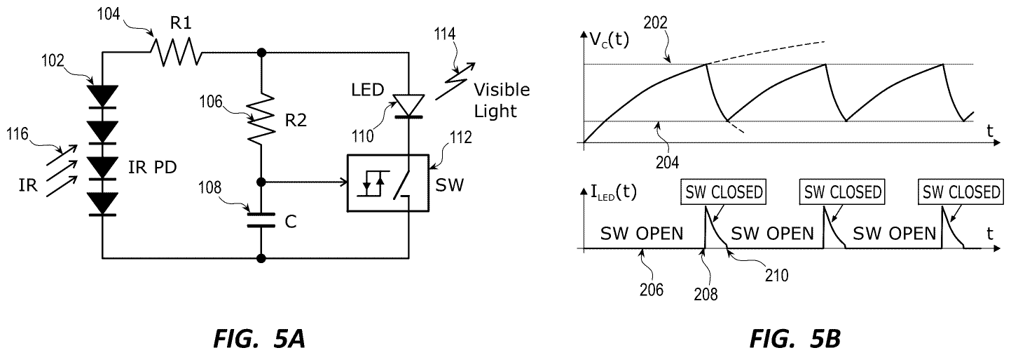

FIG. 5A illustrates a circuit diagram of one possible embodiment with a relaxation oscillator used to pulse a Light Emitting Diode (LED 110) in accordance with some embodiments. The circuitry may contain a set of serially connected photosensitive diodes (PDs 102). The PDs 102 may convert power of an IR 116 light/energy source into electrical current. In some embodiments the PDs 102 may be LEDs. In the illustrated there are four serially connected PDs 102, but the number of PDs 102 may be increased or decreased as desired or with the intended depth of the marker.

The produced electrical current passes through two resistors R1 104 and R2 106 before charging a capacitor 108. The capacitor may be connected to a visible light LED 110 and a custom switch 112 which may respond to voltage action of the control input V_{c}(t) taken from the capacitor 108. Once the switch closes, and the capacitor may discharge its stored electrical energy through the LED 110 to produce visible light 114.

The LED 110 may pulse visible light 114 due to the energy stored by the capacitor 108 being periodically applied to the LED 110. The LED 110 may be in an on state emitting visible light 114 when the capacitor 108 discharges its stored energy. The LED 110 may be in an off state and not emitting visible light 114 when the capacitor 108 is out of energy and is charging. The LED 110 may switch between an on state and an off state as controlled by the switch 112 thus producing a pulsing visible light 114. The light/energy 116 received from the light source may be infrared (IR).

In some embodiments, the LED 110 may be detachable and replaceable. This may allow a shorted LED to be replaced or different light emitters to be. In some embodiments, a user may be able to select and attach LEDs that emits a desired wavelength of light.

The response of the switch 112 to the charging of the capacitor 108 discussed herein may be visualized with reference to the voltage and current graphs illustrated within FIG. 5B. FIG. 5B illustrates the response of the switch 112 to the charging and discharging of the capacitor. The graphs illustrated of FIG. 5B illustrate a voltage V_{c}(t) oscillating between a high threshold level 202 and a low threshold level 204 and an LED current I_{LED}(t) flowing depending on if the switch 112 is open or closed.

The initially open switch 112 may close after a control input voltage V_{c}(t) reaches a high threshold level 202 and it may stay closed until the voltage drops below a low threshold level 204. When the voltage of the capacitor 108 is above the high threshold level 202 of the switch, the switch closes 208 and provides current across the LED 110 which will emit pulses of visible light as the capacitor 108 discharges its stored energy through the LED 110. When the capacitor reaches the low threshold 204, the switch may open 210 again thus allowing the capacitor 108 to be charged again as the voltage V_{c}(t) rises from the low threshold level 204 to the high threshold level 202. The open switch 210 may be seen as a new cycle of the circuit repeating the aforementioned process. This process may be repeated many times as illustrated within FIG. 5B.

In some embodiments, the high threshold level 202 and the low threshold level 204 may be set within the switch 112 before being connected within the electric circuitry. In some embodiments, the values for the high threshold level 202 and the low threshold level 204 set within the switch 112 may be permanent. In some embodiments, the values for the high threshold level 202 and the low threshold level 204 set within the switch 112 may be dynamic and may change with input from the user or may change internally as the switch 112 detects a change of voltage V_{c}(t) that satisfies the need of the LED but may possibly not satisfy the bounds of the high threshold level 202 and the low threshold level 204 thus needing a modification to be done to the low threshold level 204 and the high threshold level 202.

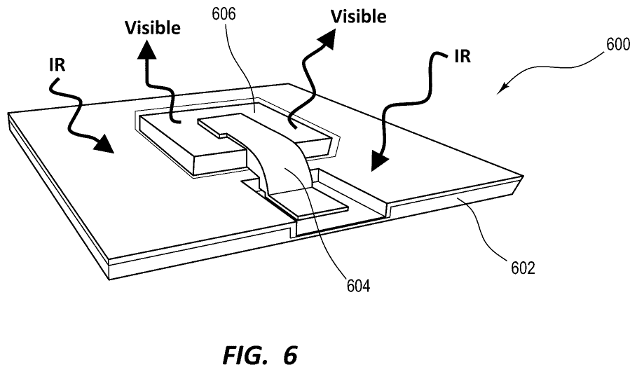

FIG. 6 illustrates a perspective view of an upconversion device 600. In some embodiments, the oscillation circuit that uses photosensitive diodes to convert light to energy to power other LEDs may be replaced or supplemented with one or more upconversion devices 600. The upconversion device 600 receives infrared light and converts it to visible light.

The upconversion device 600 may comprise one or more infrared photovoltaic diodes 602 electrically coupled to an LED 606 via a metal contact 604. The infrared light may strike the infrared photovoltaic diodes 602 which may convert the infrared light to electrical light to electrical energy which is provided to the LED 606 via a metal contact 604. The LED 606 produces a visible light. Thus, the upconversion device 600 may convert infrared light into visible light. One or more of the upconversion device 600 may be used by the markers described herein to emit light.

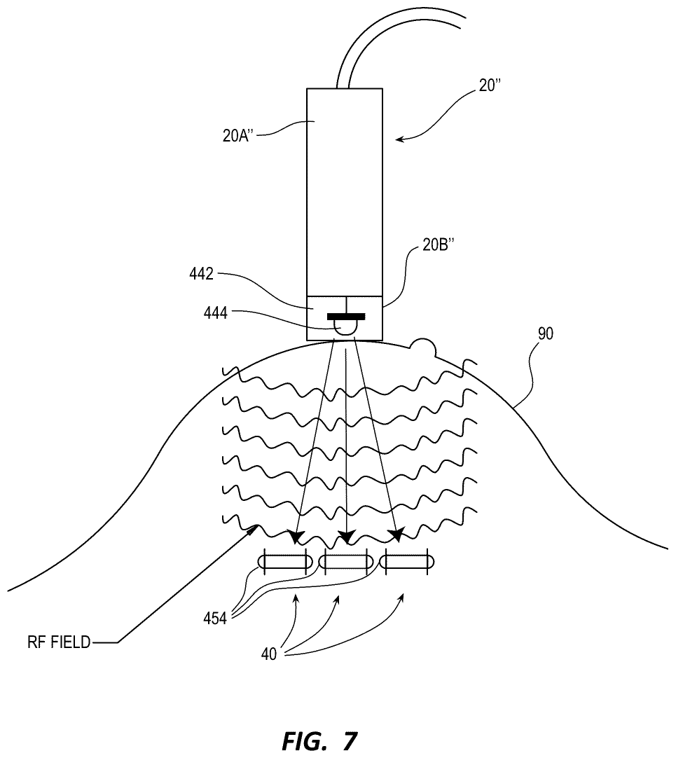

FIG. 7 is a side view of an exemplary embodiment of a probe 20 localizing a plurality of markers 40 implanted within a breast in accordance with some embodiments. The probe 20 may be a handheld device including a first or proximal end 20A configured to be held by a user, and a second or distal end 20B configured to be placed against or adjacent tissue, e.g., a patient's skin or underlying tissue.

In some embodiments, the probe 20 may use RFID to locate the markers 40. When the probe 20 is activated, it sends out a radio signal. The markers 40 may receive this signal and provide modulated backscatter to the probe 20. The probe 20 may use the modulated backscatter to identify the markers 40.

A distance from the probe 20 to the markers 40 may be estimated based on the RF signaling. In some embodiments, to estimate the distance between the probe 20 and the markers 40, the system may use a time of flight (TOF) method. In this method, the reader measures the time it takes for a radio signal to travel from the probe 20 to the marker 40 and back. The distance between the tag and the reader can then be calculated using the speed of light. In some embodiments, the system may use a received signal strength (RSS) method to determine the distance. In this method, the probe 20 may measure the strength of the radio signal received from the marker 40. The distance between the marker 40 and the probe 20 can then be estimated using a model that takes into account the factors that affect the strength of the radio signal (e.g., an RF transmission model for the breast tissue 90).

In other embodiments, the probe 20 may use other signaling techniques to identify and locate the markers 40. For example, the probe 20 may use radar rather than RFID in some embodiments.

In addition, the probe 20 may include includes a light source or light transmitter 444 configured to transmit light into tissue contacted by the distal end 20B, e.g., into breast tissue 90. In some embodiments, the light from the light transmitter 444 may be used to provide power to the markers 40.

Before the procedure, a target tissue region, e.g., a tumor or other lesion, may be identified using conventional methods. For example, a lesion (not shown) within a breast 90 may be identified, e.g., using mammography and/or other imaging, and a decision may be made to remove the lesion. A plurality of markers 40 may be implanted within the breast 90 within or adjacent the target lesion, e.g., using individual delivery devices or successively from a single delivery device (e.g., delivery device 70 of FIG. 1).

In some embodiments, the plurality of markers 40 may tested before and/or after being implanted. In some embodiments, the plurality of marker 40 may include one or more LEDs 454 that may be used to verify functionality and assist in locating implanted markers. For example, the plurality of markers 40 may be exposed to a light to verify functionality, the light may provide power to cause one or more of the LEDs 454 to illuminate. The LEDs 454 may blink or provide a constant illumination when exposed to the light. In some embodiments, the packaging within which the markers 40 are provided may prevent the markers 40 from sufficient exposure to light preventing the LEDs 454 from illuminating. In some embodiments, ambient light within a room may cause the LEDs 454 to illuminate. In some embodiments, the LEDs 454 may illuminate when struck by the light of the light transmitter 444. Additionally, after implantation, the light of the light transmitter 444 may provide sufficient energy to the markers 40 that the LEDs 454 may illuminate within the breast tissue 90. The LEDs 454 may illuminate brightly enough that the light may be seen diffusing through the breast tissue 90. The diffused light from the markers 40 may blink to provide a visual confirmation of placement and functionality of an implanted marker. In some embodiments, a marker may blink in a specific pattern or remain constantly illuminated to indicate any detected failures within the components of the marker.

Once the markers 40 are implanted, e.g., as shown in FIG. 7, the probe 20 may be activated and/or placed against a patient's skin, e.g., against the breast 90. For example, as shown in FIG. 7, the distal end 20B of the probe 20 may be placed adjacent or in contact with the patient's skin, e.g., generally above the lesion, and/or otherwise aimed generally towards the lesion and markers 40, and activated to determine a spatial relationship between the markers 40 and the distal end 20B of the probe 20, e.g., a distance and/or orientation angle, to facilitate determining a proper direction of dissection for the surgeon. The probe 20 may take multiple measurements of distance and orientation angle between the markers 40 and the probe 20 and use the multiple measurements to determine a location of the markers 40 (e.g., triangulation). Additionally, the LEDs 454 of the markers 40 may be powered by the light from the light transmitter 444 and illuminate. The illumination (e.g., blinking) may provide a visual indication to the physician of a direction or location for the probe 20 to obtain the measurements.

In some embodiments, a pre-programmed code sequence may be sent via light pulses from the light transmitter 444 of probe 20 in order to selectively activate individual markers. For example, the light pulses may be used to send a code that a marker 40 can receive via a set of diodes. A marker can recognize the voltage from the light sequence as a code to illuminate its LED. In some embodiments, the probe 20 may be able to individually address the markers via unique light pulse sequences to turn on and off the LEDs of each marker separately. In some embodiments, the light pulses may be used as a clock that the markers 40 may use for timing modulation of the switch of its antenna in addition to turning on or off the LEDs.

A display (e.g., display 34 of FIG. 1) may include a readout providing distance, angle, orientation, and/or other data based on predetermined criteria, e.g., based on the relative distance from the markers 40 to the distal end 20B of the probe 20. The distance information may be displayed as a numerical value representing the distance in units of length, such as in inches (in.) or centimeters (cm). For example, as shown in FIG. 1, a bar graph may be presented on the display 34 with the height of each bar corresponding to the distance from the respective markers 40. In some embodiments, the display 34 may present a graphical image (e.g., a two-dimensional or three-dimensional image) depicting the markers 40, the probe 20, the distance from the probe 20 to the markers 40, and/or a physiological picture of the body part containing the markers 40 (e.g., the breast).

Tissue may then be dissected, e.g., by creating an incision in the patient's skin and dissecting intervening tissue to a desired depth, e.g., corresponding to a target margin around the lesion is reached. A tissue specimen may be excised or otherwise removed using conventional lumpectomy procedures, e.g., with the markers 40 remaining within the removed specimen.

An exemplary method will now be presented describing operation of the system 10 of FIG. 1 during use. Initially, when the probe 20 is activated, the antenna may transmit an electromagnetic signal, which may be reflected by the markers 40, surrounding tissue, and/or otherwise by the patient's body. The antennas receive the reflected signals, which include crosstalk, scattering, noise, and reflections from the implanted markers 40. The received signal may be used as a baseline waveform. The markers 40 may modulate the baseline waveform, and the probe 20 may use the modulated waveform to detect and determine a distance to each of the markers. For instance, the markers 40 may switch an antenna coupled to the antenna to change the reflective properties of the antenna.

In some embodiments, the light from the probe may power the electrical circuitry of the markers 40 via the diodes and power harvesting block to support the opening and closing of the switch. In some embodiments, the diodes may be LEDs power harvesting block may repeatedly reverse the current on one or more of the diodes to cause the LEDs to blink. In some embodiments, the diodes may not be LEDs, but the energy converted by the diodes may be used to power one or more separate LEDs.

After acquisition of the baseline waveform is completed, the probe 20 may send the markers a control signal (also referred to herein as a synchronization signal) and a clock signal. In some embodiments, light pulses from a light source may be used to send the control signal and the clock signal. In some embodiments, the control signal and clock signal may be sent via signal modulation (e.g., phase). In some embodiments, the control signal may be sent via signal modulation and the clock signal may be sent via the light source. In some embodiments, the control signal may be sent via the light source and the clock signal may be sent via signal modulation.

For example, in some embodiments, the light source may be activated to generate a clock pulse. The clock pulse may comprise a plurality of light pulses in spaced-apart frames including a predetermined number of pulses(N). The light pulses may trigger the change of internal states of the markers 40 in accordance with the preprogrammed code sequence implemented in each marker 40. In response to the light pulses, the clock circuit of each marker 40 may activate the sequence generator to open and close the switch according to the code sequence to short or not short the antennas of each marker by voltage (VGl) at the gate(G) of switch parallel with the antenna, thereby modulating the backscatter signal based on the light pulses.

The clock circuit of each marker 40 may process the clock pulses. For example, the clock circuit may detect the changes in voltage output by the diodes 52 when the light pulses strike the diodes or the RF modulation. In some embodiments, the clock circuit 56 may detect clock pulses as the rising edge of the light pulses and framing events encoded as relatively long time intervals with no clocking pulses. In some embodiments, clock circuit 56 may detect clock pulses as the point at which the phase modulation on the RF signal occurs and framing events encoded as relatively long time intervals with no clock pulses.

Thus, when a frame event is detected (i.e., a relatively long period of time without a change in voltage from the diodes 52 or an RF signal modulation), the clock circuit 56 may reset the sequence generator 58 to its initial state. The clock pulses following the frame event control timing for generation of the code sequence by the sequence generator 58, represented as gi (i), which is preprogrammed in each marker 40.

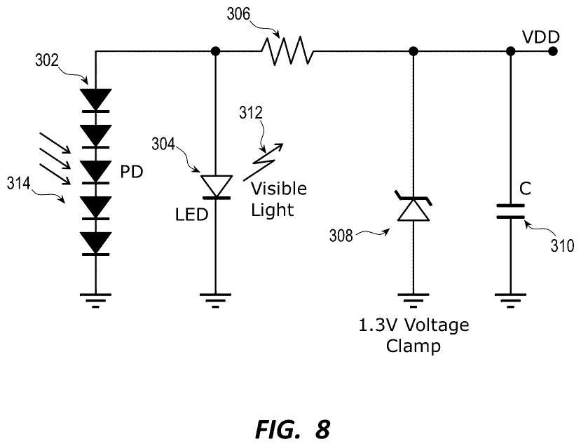

FIG. 8 illustrates a circuit diagram of one possible embodiment of a power harvesting module of a reflector and a LED to provide a visual indicator. The circuit diagram may include one array of PDs 302 in series connection, as shown in FIG. 8, or a sufficient set of such arrays connected in parallel (not shown) that may convert IR light 314 from outside sources into electrical current. The PD 302 may be connected to an LED 304 and through a resistor 306, to a 1.3V Voltage Clamp 308 and a capacitor 310. These components may reside on a semi-conductor chip, a micro-controller chip, a printed circuit board or on any other substrate that may work with electrical circuitry.

In certain embodiments, there may be only one PD array connected to the electronic circuitry as a small electrical current may be needed to power the electrical components at sufficient voltage values for VDD. In certain embodiments, the sufficient set of PDs may include more than one PD arrays in parallel as a higher electrical current may be needed to run the electrical components. In certain embodiments, the light received by the PDs 302 from the light source may be infrared (IR).

The output voltage from the PDs 302 may be regulated to control VDD voltage at required value (e.g. 1.3V) despite the larger voltage values generated by the PD array (302) during the events of high light (314) intensity. This may be achieved through the 1.3V voltage clamp 308. At higher voltages on PD 302, the voltage divider made with resistor 306 and 1.3V voltage clamp 308 connected in series may regulate the voltage of an electrical signal thus providing stable VDD power to the circuit despite the surges of high voltage events at PD array 302.

The converted electrical current generated by the PDs 302 may pass through the connected resistor 306 and into the capacitor 310 thus charging it to VDD level. When the IR 314 light intensity from the outside light source is sufficiently large it may generate enough voltage at PDs 302 and turn on the LED 304, which can emit visible light. The visible light emitted by LED 304 can be used my used for localization of the LED.

In one example, the LED 304 may emit Infrared or Ultraviolet light for sensors outside of the patient's body to detect. This may be the case in robotically preformed procedures, or procedures performed by a medical professional from a remote location that may need an indication that is more accurate than visible light such as an indication from Infrared light being picked up by a sensor.

In some embodiments, the LED 304 may be detachable and replaceable in the case of the LED 304 needing to be replaced if shorted or in the case of needing to replace the LED 304 with a different light emitter or indicator. For example, the LED 304 may be replaced by a diode that emits different wavelengths of light that may not be supported by a standard LED. In another example, a broken or shorted LED may be replaced with a new LED.

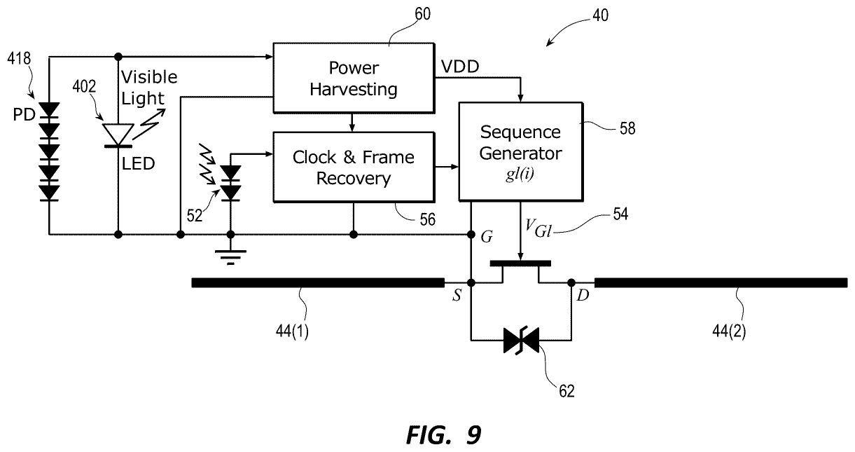

FIG. 9 illustrates a block diagram of one possible embodiment of a marker where an LED 402 is connected to a Power Harvesting module and may produce visible light as an indication to the user. The marker 40 may include one or more circuits or other electrical components encased or embedded in an electronics package and configured to modulate incident signals from the probe used to identify and/or locate the marker 40. The components may be mounted on a semiconductor chip, print circuit board (PCB), an/or other substrate carried in the package, and encased within the package such that the components are electrically isolated from one another other.

In an exemplary embodiment, the components may include an energy converter 52, IR clock detector 52′, a switch 54, a clock recovery circuit 56 coupled to the IR clock detector 52′, and sequence generator 58 coupled to the clock recovery circuit 56 and the switch 54, to generate a code sequence to open and close the switch 54 to modulate signals reflected by the marker 40 back to a probe based on a code sequence.

The marker 40 may include one or more additional components, e.g., a power harvesting circuit 60 coupled to the energy converter 52 for generating electrical energy to operate one or more electrical components of the marker 40 such as the clock recovery circuit 56 and the sequence generator 58, and/or an Electro Static Discharge (ESD) protection device 62 to provide protection against an electrostatic discharge event.

The sequence generator 58 of each marker 40 may be preprogrammed such that the code sequences generated by the sequence generators in deployed markers are orthogonal to one another, i.e., the sequence generators 58 may open and close the respective switches 54, based on the light pulses from the light source of the probe, to modulate the reflective properties of the markers 40 differently from one another, and the probe may be configured to analyze the reflected signals to identify and locate each of the markers 40 substantially simultaneously based on the resulting modulation in the reflected signals received by the probe.

The switch 54 may be a field effect transistor (FET), e.g., a junction field effect transistor (JFET), with the sequence generator 58 coupled to the gate (G) and the diodes 52, clock circuit 56, and a first antenna wire 44 (1) coupled to the drain (D). A second antenna wire 44 (2) may be coupled to the source(S) of the switch 54 to provide a pair of antennas 44 for the marker 40. In an exemplary embodiment, the switch 54 may include an enhancement mode pseudomorphic high electron mobility transistor (E—PHEMT).

In an exemplary embodiment, the energy converter 52 includes a plurality of photosensitive diodes capable of transforming incident light (e.g., infrared light) striking them into electrical energy (e.g., a predetermined minimum voltage). As shown, multiple pairs of diodes 52 may be connected in series, which may be arranged on different sides of the package. For example, given that photosensitive diodes are directional, at least two pairs of diodes 52 may be mounted within the package offset one hundred eighty degrees (180°) or otherwise relative to one another, e.g., such that at least one pair of diodes 52 may receive light from the light source of the probe regardless of the orientation of the marker 40 relative to the probe after implantation. The package may be at least partially transparent or the diodes 52 may be exposed such that light directed towards the package may be received by the diodes 52.

Light from the light pulses intermittently striking the diodes 52′ may generate a voltage that may be used by the clock circuit 56 to provide a control signal that may be used to activate the sequence generator 58 to open and close the switch 54, e.g., based on a pre-programmed code sequence. In some embodiments, a pre-programmed code sequence may be sent via the light pulses from the probe in order to selectively activate individual markers. For example, the light may be used to send a code that a marker 40 can receive via the diodes 52′. The marker can recognize the light sequence as a code to illuminate the LED 402. In some embodiments, the probe may be able to individually address the markers, via unique light pulse sequences or different wavelengths, to turn on and off the LEDs of each marker separately. In some embodiments, the light pulses may be used as a clock that the markers may use for timing modulation of the switch 54 in addition to turning on or off the LED 402.

In addition, the power harvesting block 60 may harvest electrical energy, as needed, from the diodes 52 to provide voltage and/or other electrical energy to the sequence generator 58 and/or other components of the marker 40. As a result of the sequence generator 58, the marker 40 is made to change its structure between two form factors, thereby providing a passive reflector modulation. By being able to change the switch 54 from closed to open, the reflection properties of the antennas 44 may be changed significantly and used by the probe to identify, locate, and/or distinguish the markers 40 within the patient's body.

The ESD device 62 may be coupled in parallel across the switch 54, e.g., between the drain (D) and source(S), to provide protection against an electrostatic discharge event. For example, use of an E—pHEMT device as switch 54 sets restrictions on the absolute maximal voltage between the drain (D) and source(S) and, therefore, across the marker's antennas. In the exemplary embodiment, the maximal voltage across the switch 54 may be no more than about five Volts (5 V). Modern breast surgery often involves the use of electro cutting tools, electocautery tools, and/or other tools (not shown), which can generate electrical pulses of a few kV. If such a tool gets close to the marker 40, the tool can cause a very large voltage across antenna wires 44 and destroy the switch 54.

To increase survivability of the marker 40 during operation of such tools, the ESD protection device 62 truncates voltage on the switch device when the voltage approaches the maximal value. Generally, the ESD protection device 62 in the marker 40 should have low capacitance that does not shunt the antennas 44 for the high frequency range of the UWB signal coming from the probe 20. In certain embodiments, the ESD protection device 62 may be a transient voltage suppressor, such as a Zener diode, a low-capacitance varistor, and the like. Alternatively or in addition, other ESD protection devices may be provided. For example, a capacitor (not shown) may be provided in series to one or both of the antennas 44 to provide additional ESD protection of the switch 54.

Within one embodiment, a LED 402 may be attached to the Power Harvester 60 as illustrated within FIG. 9. The LED 402 may be powered by the Photodiodes within the Power Harvester 60 circuit in a similar fashion as is described within FIG. 8. The LED may serve as an indicator that may provide visual light 418 as an indication to the user that a marker has been detected, thus localizing lesions within a patient's body. In some embodiments, the LED 402 may serve as a visual indication for a surgeon as to the location of the markers that have been placed into the patient's body. In one alternative, the LED may emit infrared light for sensors outside of the patient's body to detect. This may be the case in robotically preformed procedures, or procedures performed by a surgeon or medical staff from a remote location that may need more of an indication then only visible light.

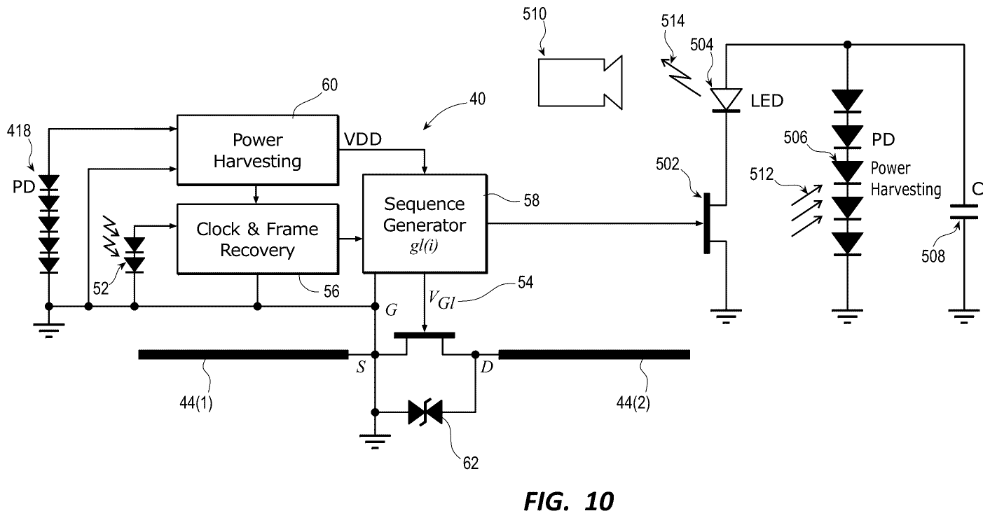

FIG. 10 illustrates a circuit diagram of one possible embodiment of a marker where orthogonal codes may be used for LED modulation and localization of the LED of the marker. Within one embodiment, an LED 504, PDs 506 and a capacitor 508 may be connected to a switch 502 which may be connected to the sequence generator 58 of the circuit diagram of the marker 40.

The sequence generator may provide the aforementioned sequence code to a switch 502 to open and close according to the sequence code thus controlling the pulsating of an LED 504. A set of PDs 506 may receive IR 512 light/energy from an outside source and may generate an electrical current. The electrical current may flow from the group of PDs 506 and into the capacitor 508 thus charging it. Once the switch 502 is activated by the sequence generator 58, the capacitor 508 may discharge its energy through the LED 504 and the LED 504 may produce visible light 514. Once the switch 502 is deactivated by the orthogonal code from the sequence generator 58, the LED 504 may turn off and power may flow into the capacitor 508 thus charging it. The opening and closing of the switch 502 may depend on the orthogonal code received from the sequence generator 58. In one alternative, the sequence generator 58 may send a constant on or off code to the switch 502 thus keeping the LED 504 in an on or off state if pulsating is not desired. In another alternative, the sequence generator 58 may send an orthogonal code that may vary how long the switch 502 is on for thus varying the length of pulses emitted by the LED 504.

In some embodiments, external photo sensors and/or an imaging device 510 may be tuned to the pulsating visible light 514 from the LED 504. In other embodiments, the LED 504 may emit an ultraviolet or IR light that the imaging device 510 may be tuned to. The imaging device 510 may receive the pulsating light from the LEDs 504 and may analyze the pulsating pattern according to but is not limited to the length of each pulse, the time between each pulse, or how fast and/or slow the LED 504 is pulsing. The blinking pattern from the LED 504 may be a code that the imaging device 510 may use to identify different markers.

The marker 40 may include one or more circuits or other electrical components encased or embedded in an electronics package and configured to modulate incident signals from the probe used to identify and/or locate the marker 40. For example, the components may be mounted on a semiconductor chip, print circuit board (PCB), and/or other substrate carried in the package, and encased within the package such that the components are electrically isolated from one another other.

In an exemplary embodiment, the components may include an energy converter (e.g., photosensitive diodes 52), a switch 54, a clock circuit 56 coupled to the energy converter 52, and sequence generator 58 coupled to the clock circuit 56 and the switch 54, to generate a code sequence to open and close the switch 54 to modulate signals reflected by the marker 40 back to a probe based on a code sequence.

The marker 40 may include one or more additional components, e.g., a power harvesting circuit 60 coupled to the photosensitive diodes 52 for generating electrical energy to operate one or more electrical components of the marker 40, e.g., the sequence generator 58, and/or an Electro Static Discharge (ESD) protection device 62 to provide protection against an electrostatic discharge event.

The sequence generator 58 of each marker 40 may be preprogrammed such that the code sequences generated by the sequence generators are orthogonal to one another, i.e., the sequence generators 58 may open and close the respective switches 54, based on the light pulses from the light source of the probe, to modulate the reflective properties of the markers 40 differently from one another, and the probe may be configured to analyze the reflected signals to identify and locate each of the markers 40 substantially simultaneously based on the resulting modulation in the reflected signals received by the probe.

The switch 54 may be a field effect transistor (FET), e.g., a junction field effect transistor (JFET), with the sequence generator 58 coupled to the gate (G) and the photosensitive diodes 52, clock circuit 56, and a first antenna wire 44 (1) coupled to the drain (D). A second antenna wire 44 (2) may be coupled to the source (S) of the switch 54 to provide a pair of antennas 44 for the marker 40. In an exemplary embodiment, the switch 54 may include an enhancement mode pseudomorphic high electron mobility transistor (E—PHEMT).

In an exemplary embodiment, the photosensitive diodes 52 includes a plurality of photosensitive diodes capable of transforming incident light (e.g., infrared light) striking them into electrical energy (e.g., a predetermined minimum voltage). As shown, multiple pairs of diodes 52 may be connected in series, which may be arranged orthogonally to one another spatially. For example, given that photosensitive diodes are directional, at least two pairs of diodes 52 may be mounted within the package offset one hundred eighty degrees (180°) or otherwise relative to one another, e.g., such that at least one pair of diodes 52 may receive light from the light source of the probe regardless of the orientation of the marker 40 relative to the probe after implantation. The package may be at least partially transparent or the diodes 52 may be exposed such that light directed towards the package may be received by the diodes 52.

Light from the light pulses intermittently striking the diodes 52′ may generate a voltage that may be used by the clock circuit 56 to provide a control signal that may be used to activate the sequence generator 58 to open and close the switch 54, e.g., based on a pre-programmed code sequence. In some embodiments, a pre-programmed code sequence may be sent via the light pulses from the probe in order to selectively activate individual markers. For example, the light may be used to send a code that a marker 40 can receive via the diodes 52′. The marker can recognize the light sequence as a code to illuminate the LED 402. In some embodiments, the probe may be able to individually address the markers via unique light pulse sequences to turn on and off the LEDs of each marker separately. In some embodiments, the light pulses may be used as a clock that the markers may use for timing modulation of the switch 54 in addition to turning on or off the LED 402.

In addition, the power harvesting block 60 may harvest electrical energy, as needed, from the diodes 52 to provide voltage and/or other electrical energy to the sequence generator 58 and/or other components of the marker 40. As a result of the sequence generator 58, the marker 40 is made to change its structure between two form factors, thereby providing a passive reflector. By being able to change the switch 54 from closed to open, the reflection properties of the antennas 44 may be changed significantly and used by the probe to identify, locate, and/or distinguish the markers 40 within the patient's body.

The ESD device 62 may be coupled in parallel across the switch 54, e.g., between the drain (D) and source(S), to provide protection against an electrostatic discharge event. For example, use of an E-pHEMT device as switch 54 sets restrictions on the absolute maximal voltage between the drain (D) and source(S) and, therefore, across the marker's antennas. In the exemplary embodiment, the maximal voltage across the switch 54 may be no more than about five Volts (5 V). Modern breast surgery often involves the use of electro cutting tools, electrocautery tools, and/or other tools (not shown), which can generate electrical pulses of a few kV. If such a tool gets close to the marker 40, the tool can cause a very large voltage across antenna wires 44 and destroy the switch 54.

To increase survivability of the marker 40 during operation of such tools, the ESD protection device 62 truncates voltage on the switch device when the voltage approaches the maximal value. Generally, the ESD protection device 62 in the marker 40 should have low capacitance that does not shunt the antennas 44 for the frequency range of the small amplitude UWB signal coming from the signals from the probe 20. In certain embodiments, the ESD protection device 62 may be a transient voltage suppressor, such as a Zener diode, a low-capacitance varistor, and the like. Alternatively or in addition, other ESD protection devices may be provided. For example, a capacitor (not shown) may be provided in series to one or both of the antennas 44 to provide additional ESD protection of the switch 54.

Within one embodiment, a LED 402 may be connected to the Power Harvester 60. The LED 402 may be powered by the Photodiodes within the Power Harvester 60 circuit in a similar fashion as described within FIG. 8. The LED may serve as an indicator that may provide visual light 418 as an indication to the user that a marker has been detected, thus localizing lesions within a patient's body. In some embodiments, the LED 402 may serve as a visual indication for a surgeon as to the location of the markers that have been placed into the patient's body. In one alternative, the LED may emit infrared light for sensors outside of the patient's body to detect. This may be the case in robotically preformed procedures, or procedures performed by a surgeon or medical staff from a remote location that may need more of an indication then only visible light.

The sequence generator 58 may have a code sequence configured to alternately open and close the switch connected to the LED of a first marker with each clock pulse, while a second marker may have a code sequence that opens and closes the switch connected to the LED with every other pulse. In one example, multiple markers may modulate their LED in a different, i.e., orthogonal, manner than each other, which the processor(s) of the probe and/or display unit may process to identify and/or locate each of the markers.

The processor(s) of the probe and/or display unit may perform separation and analysis of waveforms of visible light associated with the individual LEDs using the orthogonal code sequences and the exemplary algorithm described below.

To describe a method for the use of orthogonal sequences we consider a set of sequences in the form of s₁(i)={−1,1}, instead of gₗ(i)={0,1}, where index i=0 N−1. These sequences contain the same and even number of symbols N=2 m. They are balanced and orthogonal, i.e.,

$${\sum }_{i = 0}^{N - 1} {s}_{l} ( i ) = 0 and {\sum }_{i = 0}^{N - 1} {s}_{l} ( i ) {s}_{k} ( i ) = \{ \begin{matrix}N , & l = k \\ 0 , & l \neq k\end{matrix}$$

The processors within the probe may perform detection and localization of each marker by separating the visible light from the specific marker and performing further analysis of the visible light characteristics. This analysis may include but is not limited to using the length of time that the LED is on, the length of time between pulses, the differing lengths of time of the pulsing LED, the strength of visible light from the LED.

With the visible light separated for each marker, the processor(s) may then process the individual signals to locate the individual markers, i.e., process the separated visible light to determine a distance from the probe to the respective markers 40. This processing may be performed substantially simultaneously, allowing information regarding each of the markers 40 to be presented to the user at the same time, e.g., on the display of a display unit.

For example, each individual visible light associated with a marker may be processed initially to identify the power envelope of the visible light from the LED of the signal waveform, and then determine the time delay of the return pulse in the visible light to locate the marker. For example, to provide a distance measurement, the time delay of the returned visible light pulse may be measured with respect to the time of a time zero plane, associated with the detection of the visible light from the probe antenna inter facing the tissue, to evaluate propagation delay in the path, e.g., from the probe to the marker 40 and back to the probe, e.g. and, then the distance between the tip of the probe and the marker 40 may be calculated while taking into account the propagation speed of the visible light in the tissue.

A display system may then use this information to produce a visual localization of where each marker is located. In one example, this visual localization may be utilized by surgeons or medical experts to provide easier and more accurate surgical procedures. In another example, this visual localization may be used by medical professionals to analyze the growth or shrinkage of the tissue area where the markers are implanted. Embodiments herein may not be limited for use of medical professionals and may be utilized by all users to view the location of the one or more markers.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to an “embodiment” means that a particular feature, structure, or characteristic described in connection with that embodiment is included in at least one embodiment. Thus, references to embodiments throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

What is claimed is:

1. A marker sized for introduction into a target tissue region within a patient's body, comprising:
photosensitive diodes configured to convert external light received from a light source outside a patient's body into electrical energy;
an antenna;
a switch coupled with the antenna; and
circuitry configured to:
selectively apply the electrical energy to a light emitting diode (LED) to cause the LED to emit a light, and
selectively apply the electrical energy to the switch to open and close the switch.

2. The marker of claim 1, wherein the circuitry comprises a relaxation oscillator coupled to the one or more photosensitive diodes.

3. The marker of claim 1, wherein the circuitry is configured such that the light from the light source causes the switch to open and close to modulate electromagnetic signals from a probe reflected by the marker.

4. The marker of claim 1, wherein the circuitry comprises a storage circuit coupled to the photosensitive diodes for storing electrical energy generated by the energy converter until a predetermined threshold is achieved, wherein the storage circuit delivers the electrical energy to the LED when the predetermined threshold is achieved.

5. The marker of claim 1, wherein the circuitry comprises a sequence generator, the sequence generator configured to control application of the electrical energy to the LED based on the code sequence.

6. The marker of claim 1, wherein the external light received from the light source is infrared (IR).

7. The marker of claim 1, wherein the circuitry applies the electrical energy to the one or more of the photosensitive diodes during a first period of time, and applies the electrical energy to the switch during a second period of time.

8. The marker of claim 1, wherein the circuitry applies the electrical energy to LED and applies the electrical energy to the switch simultaneously.

9. A system for localization of a target tissue region within a patient's body, comprising:
a probe comprising one or more antennas for transmitting radiofrequency signals into a patient's body and receiving backscatter signals; and
a plurality of markers sized for introduction into a target tissue region within the patient's body, comprising:
photosensitive diodes configured to convert external light received from a light source outside a patient's body into electrical energy;
an antenna;
a switch coupled with the antenna; and
circuitry configured to:
selectively apply the electrical energy to a light emitting diode (LED) to cause the LED to emit a light, and
selectively apply the electrical energy to the switch to open and close the switch.

10. The system of claim 9, wherein the circuitry comprises a relaxation oscillator coupled to the one or more photosensitive diodes.

11. The system of claim 9, wherein the circuitry is configured such that the external light from the light source causes the switch to open and close to modulate electromagnetic signals from a probe reflected by the marker.

12. The system of claim 9, wherein the circuitry comprises a storage circuit coupled to the photosensitive diodes for storing electrical energy generated by the energy converter until a predetermined threshold is achieved, wherein the storage circuit delivers the electrical energy to the LED when the predetermined threshold is achieved.

13. The system of claim 9, wherein the circuitry comprises a sequence generator, the sequence generator configured to control application of the electrical energy to the LED based on the code sequence.

14. The system of claim 9, wherein the circuitry applies the electrical energy to the LED during a first period of time, and applies the electrical energy to the switch during a second period of time.

15. The system of claim 9, wherein the circuitry applies the electrical energy to LED and applies the electrical energy to the switch simultaneously.

16. The system of claim 9, further comprising an imaging device configured to detect the light from the LED, analyze a blinking pattern from the LED, and identify a marker based on the blinking pattern.

17. A method for localization of a target tissue region within a patient's body, comprising:
introducing a marker into the target tissue region, the marker comprising:
photosensitive diodes configured to convert external light from a probe outside a patient's body into electrical energy;
an antenna;
a switch coupled with the antenna; and
circuitry configured to:
selectively apply the electrical energy to a light emitting diode (LED) to cause the LED to emit a light, and
selectively apply the electrical energy to the switch to open and close the switch;
placing the probe adjacent the target tissue region; and
activating the probe, whereupon the probe transmits the external light to the marker causing the LED of the marker to emit the light.

18. The method of claim 17, wherein the circuitry of the marker comprises a relaxation oscillator coupled to the one or more photosensitive diodes.

19. The system of claim 17, wherein the circuitry of the marker is configured such that the external light from the probe causes the switch to open and close to modulate electromagnetic signals from a probe reflected by the marker.

20. The system of claim 17, wherein the circuitry comprises of the marker a storage circuit coupled to the photosensitive diodes for storing electrical energy generated by the energy converter until a predetermined threshold is achieved, wherein the storage circuit delivers the electrical energy to the LED when the predetermined threshold is achieved.
